(19) United States
(12) Patent Application Publication
(10) Pub. No.: US 2021/0035663 A1
(43) Pub. Date: Feb. 4, 2021

(54) FACILITY AND METHOD FOR VALIDATING THE ORIGIN AND/OR QUALITY OF BIOLOGICAL SPECIMEN SECTIONS

(71) Applicant: DREAMPATH DIAGNOSTICS, Strasbourg (FR)

(72) Inventors: Pablo JORDAN NAVAS, Madrid (ES); Valerie WILHELM, Eschau (FR)

(21) Appl. No.: 16/930,588

(22) Filed: Jul. 16, 2020

(30) Foreign Application Priority Data
Jul. 29, 2019 (FR) 19 08590

(51) Int. Cl. G16H 10/40; G01N 33/483

(52) U.S. Cl. CPC G16H 10/40; G01N 33/4833

(57) ABSTRACT

The facility for validating the origin and/or quality of biological specimen sections, stored on specimen slides, includes a device for reading encoded identification data, a device for acquiring biometric data specific to a biological specimen, and a data transmitter to a computer processor. The facility validates the origin and/or the quality of biological specimen slides or validates the quality of the staining of biological specimen slides. There are methods for implementing the facility in the context of such uses.

### CROSS-REFERENCE TO RELATED APPLICATIONS

See Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the Invention

The present invention relates to a facility for validating the origin and/or quality of biological specimen sections, stored on specimen slides bearing encoded identification data and taken from paraffin blocks also bearing encoded identification data.

### 2. Description of Related Art Including Information Disclosed Under 37 CFR 1.97 and 37 CFR 1.98.

In the context of the medical care of a patient or of research work in the human, animal or plant fields, tissue or cell samples are commonly taken for histological and/or molecular analysis, which may or may not be intended to prepare a diagnosis.

In order to allow perfect viewing under the microscope of the cell or tissue structures being analyzed, such samples are subject to prior preparation comprising different steps.

In short, during the latter, the freshly sampled specimens are placed in open cassettes, undergo a dehydration phase, then are included in paraffin, resulting in blocks of biological specimens. Fine and regular sections, from 4 to 10 μm thick, taken from the latter, are next placed on specimen slides, undergo various other treatments using chemical products and stains in order to expose the structures to be observed, and are lastly covered with a glue, then a strip allowing them to be read under the microscope and providing them with protection.

It is worth noting the due to the aging of the population and the increased incidence rate of certain diseases such as cancer, today pathology laboratories have reason to manage a very large number of biological specimens, for example in the form of biopsies, or excised tissues.

Biopsies are generally small, or even very small, and generally lead, after treatment and dehydration, to a single or very small number of paraffin blocks. On the contrary, excised tissues can be large, in particular when they consist of a whole organ, such that it may be necessary to split them, which then leads to multiple paraffin blocks each incorporating one of the obtained fractions.

All of the steps in the preparation of the biological specimens for analysis assume many manipulations, during which measures must be implemented to guarantee not only the accuracy of the analyses done and in particular of the diagnoses made, but also a correspondence between their actual origin and that which is attributed to them during their journey.

Thus, when it is collected, each biological specimen is assigned a unique reference number, provided by the computer systems of the laboratories, linking it to the patient from which it originates, and which will remain with it throughout its entire journey, whether it is stored in a jar, a cassette, a paraffin block or on a specimen slide. Furthermore, each specimen (biopsy or excised tissue fragment) has a unique form, sometimes detectable by the naked eye, but in other cases only by microscopic analysis.

At this time, the operation consisting of verifying whether the reference number of a specimen slide, assumed to bear a biological specimen coming from a section of a given paraffin block, matches the reference number of the latter and therefore comes from the same patient, is primarily done by visual validation. As an extra precaution, and when the specimen allows it, a visual inspection is sometimes done to determine whether the shape of the specimen placed on a specimen slide corresponds to that of the specimen integrated into the paraffin block.

However, such a method is currently reaching its limits, due to the ever-increasing volumes of specimens to be managed by anatomical pathology laboratories, and may lead to errors, in particular when assigning reference numbers to the specimen slides when they are produced.

In some cases, pathologists are fortunately able to detect such errors during their procedures, in particular if the profile of the patient to whom a slide has been assigned does not match that of the specimen stored on that same slide, or if the shape of the specimen stored on the slide differs from that of the specimen included in the block. Nevertheless, the laboratory must then look for the original paraffin block, re-cut it, stain it, etc., which requires additional working time and cost, and above all a delay in diagnosing the patient. Additionally, when on the contrary the error is impossible to detect, for example because the profile of the corresponding patient, identified using the reference number borne by the specimen slide, may be compatible with the analyzed biological specimen, the pathologist will make a diagnosis that will unfortunately be delivered to an incorrect patient. This can of course be fatal, both for the latter if he then receives inappropriate care and/or treatment having side effects, and for the patient actually at the origin of the analyzed sample, who will then not receive the diagnosis or any care.

Furthermore, it has been observed that diagnostic errors can result in a lack of quality of the analyzed specimen slides.

Indeed, the production of the latter is especially delicate, requires great dexterity by the technicians in question, and can be affected by many parameters, such as poor quality of the slide used, technician fatigue, the ambient temperature, etc. The section may also not be taken deeply enough in the paraffin block, such that a part of the specimen that may be of major interest in establishing the diagnosis is ultimately absent on the obtained specimen slide.

A poor quality of the specimen slide may further result from an approximate dying of the biological specimen stored thereon, using chemical solutions with degraded properties. Due to the very large number of specimen slides stained each day, the quality of the reagents and consumables present in the baths in fact decreases as they are used. Thus, if the stains, alcohols and other chemical products are not changed and refreshed regularly, the quality of the staining in terms of sharpness, contrast, intensity of the color, decreases after each treated batch of slides. Yet this decrease in staining quality of a specimen slide is very difficult to detect with the naked eye, since it occurs very gradually. Another readability defect may be due to the absence of correct spreading of the glue on the section, making reading with the microscope impossible. This low quality of the slides due to a negligent preparation can make the diagnoses done by the pathologist more difficult, or even cause incorrect diagnoses, certain details or particularities of the tissues not having been revealed and therefore remaining undetectable.

### BRIEF SUMMARY OF THE INVENTION

The aim of the present invention is to propose a solution making it possible to address all of the problems described above, and therefore to guarantee the reliability of the diagnoses established by pathology laboratories, both in terms of precision and in terms of recipient, in particular through a double validation regarding the origin of the biological specimens stored on specimen slides, done immediately after the production of the latter. Additionally, the invention also aims to allow a validation of the quality of the specimen slides when they are produced, making it possible to send the pathologists responsible for analysis and diagnosis operations only specimen slides having the required preparation quality.

To that end, the present invention relates to a facility for validating the origin and/or the quality of biological specimen sections, taken from paraffin blocks provided with encoded identification data and stored on specimen slides also provided with encoded identification data, said facility comprising:

at least one device for reading encoded identification data, characterized in that it further comprises:

at least one device for acquiring biometric data specific to a biological specimen included in a paraffin block or specific to a biological specimen stored on a specimen slide in the form of a section taken from a paraffin block,

means for transmitting read identification data and acquired biometric data to computer processing means incorporating a software application executed in order to

record, in an associated manner in memory means, the identification data read on a paraffin block and the biometric data specific to the specimen included in said paraffin block and previously acquired,

record the identification data read on a specimen slide and compare them with those recorded for said paraffin block, and, if applicable

record the biometric data specific to the biological specimen stored in section form on said specimen slide and compare them with the biometric data recorded in an associated manner with said identification data of said paraffin block, and

activate means for genesis of a signal representative of the result of the comparison(s).

According to a preferred variant embodiment, the facility according to the invention comprises an apparatus having a housing configured to receive at least one paraffin block or at least one specimen slide and in which are appropriately installed at least one device for reading encoded data intended to read the identification data of a paraffin block, at least one second device for reading encoded data intended to read the identification device of a specimen slide, as well as at least one device for acquiring biometric data intended to acquire the biometric data specific to a biological specimen included in a paraffin block and/or the biometric data specific to a biological specimen stored in section form on a specimen slide.

Another variant embodiment may further be considered in which said apparatus comprises a device for acquiring biometric data intended to acquire the biometric data specific to a biological specimen included in a paraffin block and a device for acquiring biometric data intended to acquire the biometric data specific to a biological specimen stored in section form on a specimen slide.

The facility additionally comprises such an apparatus, a computer provided with computer processing means and at least one user interface, said computer being connected to said apparatus by a wired or wireless connection.

According to another conceivable option, the apparatus itself incorporates the computer processing means and at least one user interface.

Preferably, the device(s) for acquiring biometric data is/are defined by a device for acquiring data relative to a two-dimensional or three-dimensional shape specific to a biological specimen included in a paraffin block or specific to a biological specimen stored, in the form of a section taken from a paraffin block, on a specimen slide.

Additionally, the facility according to the invention is further characterized in that it comprises at least one barcode reader making it possible to read the encoded identification data affixed on a paraffin block or on a specimen slide.

It has further been provided that the user interface preferably consists of a screen.

The invention also relates to a method for validating the origin and/or quantity of biological specimen sections, taken from paraffin blocks provided with encoded identification data, said sections being stored on specimen slides also provided with encoded identification data, using a facility as described above, characterized by the performance, in order, of the following steps:

reading the identification data of at least one paraffin block using a device for reading encoded data,

acquiring the biometric data specific to the biological specimen included in this same paraffin block using a device for acquiring biometric data,

recording, in an associated manner, the identification data of the paraffin block and the biometric data specific to the biological specimen included in this same paraffin block in memory means,

reading the identification data of at least one specimen slide using a device for reading encoded data,

acquiring the biometric data specific to the biological specimen stored in section form in said specimen slide using a device for acquiring biometric data,

comparing the identification device of said specimen slide and the identification data of said paraffin block, then comparing the biometric data specific to the biological specimen stored in section form in said specimen slide and the biometric data specific to the biological specimen included in said paraffin block

receiving a signal representative of the result of the comparisons done, and, depending on the case

Confirming the origin and/or the quality of the specimen stored on the specimen slide, or

Invalidating the origin and/or the quality of the specimen stored on the specimen slide.

Additionally, the invention also relates to a method for validating the quality of the staining of a specimen slide using a facility as previously described, characterized by the performance of the following steps:

reading the identification data of at least one specimen slide, called “reference specimen slide,” using a device for reading encoded data,

acquiring the biometric data specific to the biological specimen stored in suitably stained section form in the “reference” specimen slide using a device for acquiring biometric data,

recording, in an associated manner, the identification data of the “reference” specimen slide and the biometric data specific to the biological specimen stored in suitably stained section form in the “reference” specimen slide, in memory means,

reading the identification data of a specimen slide using a device for reading encoded data,

acquiring the biometric data specific to the biological specimen stored in stained section form in said specimen slide using a device for acquiring biometric data,

receiving a signal representative of the result of a comparison between the biometric data specific to the biological specimen stored in stained section form in said specimen slide and the biometric data specific to the biological specimen stored in suitably stained section form in said “reference” specimen slide, done using computer processing means, and, depending on the case

Confirming the quality of the staining of the biological specimen stored in stained section form in the specimen slide and keeping the specimen slide (3) as is, or

Invalidating the quality of the staining of the biological specimen stored in stained section form in the specimen slide and subjecting the specimen slide to a new staining step, or destroying the specimen slide.

The invention also relates to the use of the facility as described above to validate the origin and/or the quality of biological specimen sections, taken from paraffin blocks and stored on specimen slides or to validate the staining of biological specimen sections stored on specimen slides.

### BRIEF DESCRIPTION OF THE SEVERAL VIEWS OF THE DRAWINGS

The present invention will be better understood upon reading the description done in reference to the appended figures, provided as non-limiting examples.

FIG. 1 shows a perspective view of a paraffin block incorporating a biological specimen.

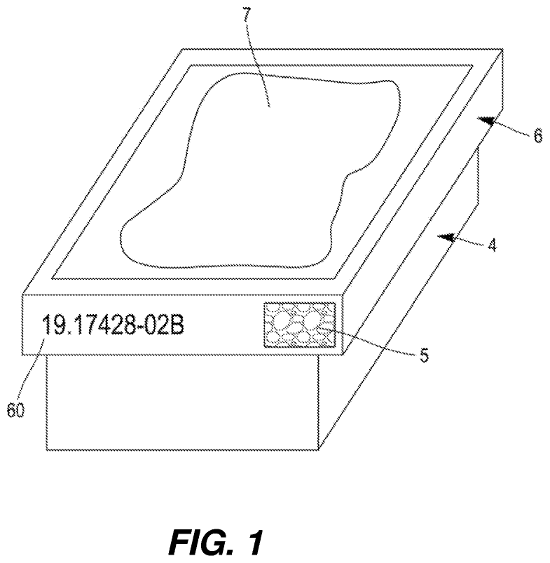

FIG. 2 is a schematic view of a specimen slide on which a section is stored taken from the paraffin block of FIG. 1.

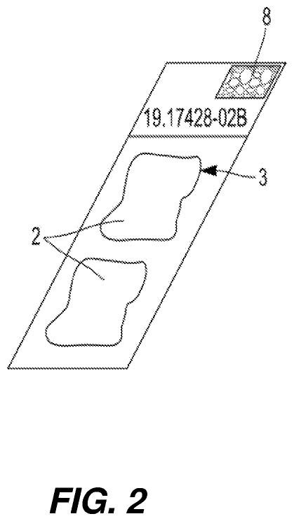

FIG. 3 is a schematic view of a variant embodiment of the facility according to the invention during a step of the method for validating the origin and/or the quality of a biological specimen section taken from the paraffin block of FIG. 1 and stored on the specimen slide of FIG. 2.

### DETAILED DESCRIPTION OF THE INVENTION

In reference to the figures, the present invention relates to a facility 1 for validating the origin and/or quality of biological specimen sections 2 stored on specimen slides 3, and taken from paraffin blocks 4.

Conventionally, identification data, representative of the patient in question, are assigned once they are taken from all of the biological specimens intended to perform the analyses and/or establish diagnoses, and will stay with them throughout their entire journey through the various departments that may be involved, irrespective of the type of medium on which they are stored.

Thus, each paraffin block 4 is provided with such identification data, generally affixed on one face 60 of a cassette 6 containing it, said data for example assuming the form of a matrix code 5, and making it possible to link each paraffin block 4 to the patient from whom the biological specimen 7 that it incorporates originates. Of course, all of the paraffin blocks 4, each incorporating a fraction of an organ coming from a single and same patient, are meant to bear a single matrix code 5.

Likewise, each specimen slide 3 on which a sampled section 2 is stored on a paraffin block 4 bears unique identification data, for example in the form of a matrix code 8. Thus, in order to guarantee that a diagnosis is sent to the patient from whom the sample originates, the matrix code 8 of a specimen slide 3 made from a paraffin block 4 must necessarily correspond to the matrix code 5 affixed on the latter.

The implementation of the facility 1 according to the invention during the manufacture of specimen slides 3 makes it possible to avoid or detect any affixing of an incorrect matrix code 8 on the latter.

In other words, the implementation of the facility 1 immediately after the production of specimen slides 3 makes it possible only to provide pathologists responsible for analyses with specimen slides 3 for which the identity of the patient has been validated beforehand.

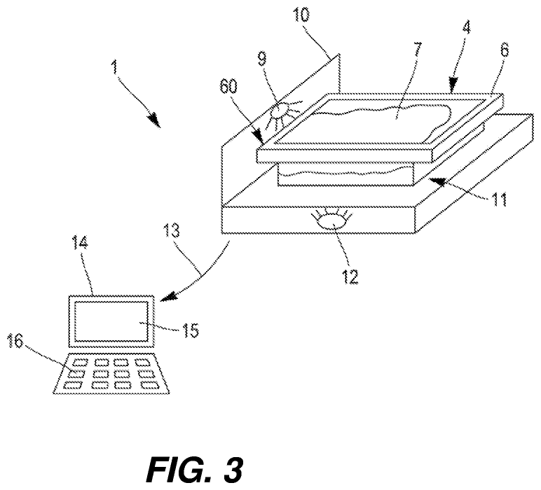

To that end, in the variant embodiment illustrated in FIG. 3, the facility comprises an apparatus 10 having a housing 11 configured to receive at least one paraffin block 4 or at least one specimen slide 3 and in which have been appropriately installed a first barcode reader 9 intended to read the matrix code 5 affixed on one face 60 of a cassette 6 containing a paraffin block 4, and a second barcode reader 12 intended to read the matrix code 8 of a specimen slide 3. Such an apparatus can be defined by a device for acquiring and analyzing images at the appropriate resolution.

Furthermore, the apparatus 10 further incorporates at least one device (not illustrated) for acquiring data relative to a two-dimensional or three-dimensional shape specific to a biological specimen 7 included in a paraffin block 4 or specific to a biological specimen 7 stored, in the form of a section 2 taken from a paraffin block 4 on a specimen slide 3.

In short, the apparatus 1 is equipped with means allowing it to read a barcode affixed on a paraffin block 4, means allowing it to read a barcode affixed on a specimen slide 3, means allowing it to determine a characteristic shape of a biological specimen 7 stored in a paraffin block 4, and means allowing it to determine a characteristic shape of a biological specimen 7 stored on a specimen slide 3, in section 2 form taken from this same block 4.

The facility 1 also comprises a computer 14, provided with a screen 15 and a keyboard 16, and connected to the apparatus 1 by data transmission means 13, which may be wired or wireless.

According to the invention, the computer 14 incorporates computer processing means incorporating a software application executed in order, in the described embodiment, to:

record, in an associated manner in memory means, the identification data read from the matrix code 5 on a paraffin block 4 and the data relative to the characteristic shape of the biological specimen 7 included in this same paraffin block 4,

record the identification data read from the matrix code 8 on a specimen slide 3 and compare them with those recorded for said paraffin block 4, and, if applicable

record the data relative to the characteristic shape of the biological specimen 7 stored in section 2 form on said specimen slide 3 and compare them with the data relative to the characteristic shape of the biological specimen 7 included in the paraffin block 4 recorded in an associated manner with the identification data of said paraffin block 4, and

activate means for genesis of a signal representative of the result of the comparison(s), for example in the form of a visual signal appearing on the screen 15 of the computer 14.

In practice, the facility according to the invention can be used in the context of the application described above to automatically validate the origin of a biological specimen 7 stored in section 2 form on a specimen slide 3.

This validation is advantageously done on two levels, namely through a comparison between the identification data affixed on the specimen slide 3 and those affixed on the paraffin block 4 used to produce it, and a second comparison done this time between the shape of the specimen 7 supported by the slide 3 and that of the specimen 7 included in the paraffin block 4. Comparing the shapes advantageously also makes it possible to detect any section defects (such as tissue folds or slide marks, etc.), and in particular sections done at insufficient depths in the paraffin block and which may lead to incorrect diagnoses.

In this context, the implementation of the facility 1 consists of first placing a paraffin block 4 in the housing 11 of the apparatus 10 of the facility 1, then reading the matrix code 5 of a paraffin block 4 using a barcode reader 9 and next determining a shape specific to the biological specimen 7 included in this same paraffin block 4 using a device for acquiring and analyzing an image at the appropriate resolution (not illustrated).

The read identification data of the paraffin block 4 and the data relative to the shape specific to the biological specimen 7 included in this same paraffin block 4 are then automatically recorded in an associated manner in the memory means of the computer 14.

Then, the paraffin block 4 is removed from the housing 11 of the apparatus 10, and replaced by a specimen slide 3 produced from said paraffin block 4, and the origin and quality of which must be validated. To that end, it is then necessary to read the identification data of the specimen slide 3, using the barcode reader 12, then to determine a shape specific to the biological specimen 7 stored in section form 2 in said specimen slide 3 using said image acquisition and analysis device.

The computer processing means of the computer 14 then generate a signal representative of the result of a comparison between the matrix code 8 of said specimen slide 3 and the matrix code 5 of said paraffin block 4 and, if applicable, a comparison between the shape specific to the biological specimen 7 stored in section form 2 in said specimen slide 3 and the shape specific to the biological specimen 7 included in said paraffin block 4, done by computer processing means. This signal is displayed on the screen 15 and, depending on the case, will correspond to a confirmation of the origin and/or of the quality of the specimen slide 3, or to an invalidation of the origin and/or the quality of the specimen slide 3.

If an insufficient match is detected between the shape of the specimen 7 present on the specimen slide 3 and that of the specimen 7 included in the paraffin block 5, or if a section 2 done at an insufficient depth is detected, the operator having produced the specimen slide 3 will be notified immediately, as of the end of this production. This will allow him to detect any error in assigning a slide 3 to an incorrect patient or to observe any production defect requiring new additional sections.

A comparison of the shapes can also make it possible to detect any tissue folds or microtome slide marks present in a biological specimen stored in a section 2 on a specimen slide 3, and which may also lead to diagnostic errors. The specimen slides 3 bearing such sections 2 will thus also be able to be removed during the validation done using the facility 1, without being sent to the pathologist responsible for analyzing them.

It is also possible to provide for the destruction of any specimen slide 3 once an error of any kind is detected, so as to avoid any future diagnostic error or assignment of the latter to an incorrect recipient.

Furthermore, the facility 1 could also be implemented solely to verify the quality of the staining of a section 2 stored on a specimen slide 3. In this case, it is provided to use a so-called “reference” specimen slide for which it is established that all of the elements that may be observed on the specimen 7 have indeed been revealed following the performed chemical treatments.

In the context of this application, it is in practice provided to first position, in the housing 11 of the apparatus 10, a “reference” specimen slide in order to acquire the shape specific to a biological specimen 7 stored in suitably stained section 2 form in this same “reference” specimen slide, using the image acquisition and analysis device, after having read the matrix code of said reference slide. The obtained image is then recorded in the memory means of the computer 14, associated with the identification data of the reference specimen slide. Then the “reference” specimen slide is removed from the housing 11 and replaced by the specimen slide 3 for which one wishes to verify the staining quality. The barcode reader 12 is activated again, then the image acquisition and analysis device is addressed, this time to determine the shape specific to the biological specimen 7 stored in stained section 2 form in the specimen slide 3.

A signal is then displayed on the screen 15 of the computer 14. It is representative of the result of a comparison between the shape specific to the biological specimen 7 stored in stained section 2 form in the specimen slide 3 and the shape specific to the biological specimen 7 stored in suitably stained section 2 form in the “reference” specimen slide, done by the computer processing means of the computer 14. This signal clearly informs the operator of the actions to be performed, namely, depending on the case

to keep the specimen slide 3 as is, when the staining proves to be satisfactory, or

to subject the specimen slide 3 to a new staining step with a refreshed solution, or

to destroy the specimen slide 3 when it proves to be irrecoverable.

1. A facility for validating the origin and/or quality of biological specimen sections, stored on specimen slides bearing encoded identification data and taken from paraffin blocks also bearing encoded identification data, said facility comprising
at least one device for reading encoded identification data, characterized in that it further comprises:
at least one device for acquiring biometric data specific to a biological specimen in a paraffin block or specific to a biological specimen stored on a specimen slide in the form of a section taken from a paraffin block,
means for transmitting read identification data and acquired biometric data to computer processing means incorporating a software application executed in order to
record, in an associated manner in memory means, the identification data read on a paraffin block and the biometric data specific to the biological specimen included in said paraffin block and previously acquired,
record the identification data read on a specimen slide and compare them with those recorded for said paraffin block, and, if applicable
record the biometric data specific to the biological specimen stored in section form on said specimen slide and compare them with the biometric data recorded in an associated manner with said identification data of said paraffin block, and
activate means for genesis of a signal representative of the result of the comparison(s).

2. The facility according to claim 1, further comprising:
an apparatus having a housing configured to receive at least one paraffin block or at least one specimen slide and in which are appropriately installed at least one device for reading encoded data intended to read the identification data of a paraffin block, at least one second device for reading encoded data intended to read the identification device of a specimen slide, as well as at least one device for acquiring biometric data intended to acquire the biometric data specific to a biological specimen included in a paraffin block and/or the biometric data specific to a biological specimen stored in section form on a specimen slide.

3. The facility according to claim 2, wherein said apparatus comprises a device for acquiring biometric data intended to acquire the biometric data specific to a biological specimen in a paraffin block and a device for acquiring biometric data intended to acquire the biometric data specific to a biological specimen stored in section form on a specimen slide.

4. The facility according to claim 2, further comprising: a computer provided with computer processing means and at least one user interface, connected to said apparatus by a wired or wireless connection.

5. The facility according to claim 2, wherein said apparatus incorporates the computer processing means and at least one user interface.

6. The facility according to claim 1, wherein the device(s) for acquiring biometric data is/are defined by a device for acquiring data relative to a two-dimensional or three-dimensional shape specific to a biological specimen included in a paraffin block or specific to a biological specimen stored on a specimen slide in the form of a section taken from a paraffin block.

7. The facility according to claim 1, further comprising: at least one bar code reader.

8. The facility according to claim 4, wherein the user interface of comprises a screen.

9. A method for validating the origin and/or quantity of biological specimen sections, taken from paraffin blocks provided with encoded identification data, said sections being stored on specimen slides also provided with encoded identification data, the method comprising the steps of:
using a facility according to claim 1,
reading the identification data of at least one paraffin block using a device for reading encoded data,
acquiring the biometric data specific to the biological specimen included in this same paraffin block using a device for acquiring biometric data,
recording, in an associated manner, the identification data of the paraffin block and the biometric data specific to the biological specimen included in this same paraffin block in memory means,
reading the identification data of at least one specimen slide using a device for reading encoded data,
acquiring the biometric data specific to the biological specimen stored in section form in said specimen slide using a device for acquiring biometric data,
comparing the identification device of said specimen slide and the identification data of said paraffin block, then comparing the biometric data specific to the biological specimen stored in section form in said specimen slide and the biometric data specific to the biological specimen included in said paraffin block,
receiving a signal representative of the result of the comparisons done, and, depending on the case
Confirming the origin and/or the quality of the specimen slide, or
Invalidating the origin and/or the quality of the specimen slide.

10. A method for validating the quality of the staining of a specimen slide, the method comprising the steps of:
using a facility according to claim 1,
reading the identification data of at least one specimen slide, called “reference specimen slide,” using a device for reading encoded data,
acquiring the biometric data specific to the biological specimen stored in suitably stained section form in the “reference” specimen slide using a device for acquiring biometric data,
recording, in an associated manner, the identification data of the reference specimen slide and the biometric data specific to the biological specimen stored in suitably stained section form in the reference specimen slide, in memory means,
reading the identification data of a specimen slide using a device for reading encoded data,
acquiring the biometric data specific to the biological specimen stored in stained section form in said specimen slide using a device for acquiring biometric data,
receiving a signal representative of the result of a comparison between the biometric data specific to the biological specimen stored in stained section form in said specimen slide and the biometric data specific to the biological specimen stored in suitably stained section form in said “reference” specimen slide, done using computer processing means, and, depending on the case
Confirming the quality of the staining of the biological specimen stored in stained section form in the specimen slide and keeping the specimen slide as is, or
Invalidating the quality of the staining of the biological specimen stored in stained section form in the specimen slide and subjecting the specimen slide to a new staining step, or destroying the specimen slide.

11. A use of the facility according to claim 1, so as to validate the origin and/or the quality of biological specimen sections, taken from paraffin blocks and stored on specimen slides or to validate the staining of biological specimen sections stored on specimen slides.
